(19) United States
(12) Patent Application Publication
(10) Pub. No.: US 2013/0158483 A1
(43) Pub. Date: Jun. 20, 2013

(54) DIALYSIS NEEDLE SYSTEM

(71) Applicant: Martin Senitko, Biloxi, MS (US)

(72) Inventor: Martin Senitko, Biloxi, MS (US)

(21) Appl. No.: 13/407,143

(22) Filed: Feb. 28, 2012

Related U.S. Application Data
(60) Provisional application No. 61/441,379, filed on Mar. 1, 2011.

(51) Int. Cl. A61M 5/158

(52) U.S. Cl. CPC A61M 5/1582
USPC 604/174; 604/272

(57) ABSTRACT

A needle for the cannulation of a dialysis access graft that is less traumatic to the graft, and less likely to dislodge.

### BACKGROUND

Hemodialysis is a medical procedure that is used to treat people with a very poor kidney function. The procedure involves circulating patient's blood through a machine with a hemodialysis filter where unwanted waste products are removed from the bloodstream. During the procedure, blood flows from the patient, via the machine, and back to the patient. Typical hemodialysis session lasts approximately 4 hours. It can also be performed over 6-8 hours or longer depending on a clinical situation. Sometimes it is performed outside of a medical facility, such as by the patient at home.

Adequate vascular access to the patient's circulation is required for a hemodialysis. It provides desired blood flow rates and can be used repeatedly to connect the patient to a hemodialysis machine. Typically, vascular access in a long-term dialysis patient is a surgically created connection (or conduit) between patient's artery and vein. It lies beneath the skin and it is cannulated with dialysis needles each dialysis session.

A native fistula (or AV fistula) is an access that is surgically created when a vein is connected directly with an artery. Vein subsequently enlarges as it adapts to high blood flow provided by an artery. This enlarged segment is then available for cannulation. A graft (or AV graft) is an access that is surgically created when an artificial vessel made of synthetic material (e.g. polytetrafluoroethylene) is used to connect a vein with an artery. This synthetic segment is then used for cannulation with dialysis needles.

Two needles are used during hemodialysis. The first needle establishes arterial connection (so-called arterial needle) and provides blood flow from an access to a dialysis machine. The second needle establishes venous connection (so-called venous needle) and provides blood flow from a dialysis machine back to dialysis access. Both needles have to remain within the access for the whole duration of treatment. Despite many advances in the field of hemodialysis since its inception, there have been no major changes in the design of dialysis needles. Currently used needles have a geometry of a circular cylinder with a circular inside lumen. They are 14 to 17 gauge thick and 1 to 1.5 inches long. They are made of surgical grade steel and thus are rigid and non-flexible. The tip of a needle has beveled surface with single opening and a cutting edge allowing penetration into the tissues. At the other end, steel part of a needle is molded into a plastic hollow cylinder that allows connection to a plastic tubing which carries blood to and from dialysis machine. Ordinarily, to secure needles in place, a piece of a medical tape is placed over each needle and the patient is asked to hold still the part of body where the access is located.

Dialysis needles can also be made rounded on the top making the bevel relatively dull. Use of such “blunt” needles requires native fistula and a presence of a scar tissue tunnel track. This typically forms after repeated cannulations using sharp needles in the exactly same place, under exactly the same insertion angle.

There are drawbacks related to the use of current dialysis needles, and improved needles would improve the dialysis process. Ideally, needles would need to remain within the access motionless throughout the entire procedure. It is, however, virtually impossible for the patient to remain completely still during the entire hemodialysis session. Consequently, as the patient moves, the sharp tip of a needle can bounce back and forth against the inner wall of the vascular access causing mechanical trauma. Depending on the degree of trauma, it can lead to several major access complications. This includes thrombus formation and eventual clotting of the entire access, formation of pseudoaneurysm, and bleeding into tissues surrounding the access with consequent hematoma formation.

Because arterial needle generates relatively high amount of negative pressure, it can make the inner wall of an access to be pulled against the tip of a needle. Outside of possible wall trauma, this effect prevents adequate blood flow through the entire system. It leads to undesired breaks in the procedure, frequent needs to reposition the needle, and ultimately inadequate dialysis. In turn, venous needle returning the blood to an access generates relatively high degree of positive pressure which transmits to the walls of an access and can, by a recoil force, push the needle out from the access.

### SUMMARY

The invention presents a unique design of a hemodialysis needle. This design accomplishes the following. Firstly, it reduces mechanical trauma inflicted by the sharp tip of the needle to the walls of the access during the hemodialysis and thus decreases the risk of complications such as hematoma, thrombosis, and loss of access. Secondly, it reduces the risk of inadvertent needle withdrawal from dialysis access during the hemodialysis. Thirdly, it improves the stability of blood flow through the needle by reducing the chances of adherence of the needle opening against access walls.

The dialysis needles of the invention have a mechanical bulb like feature that may be deployed while the needle is in the access or graft. These needles with a blunt feature support high blood flows and although they may “bounce” they minimize trauma to the graft which is advantageous. They are also much less likely to dislodge and therefore they are a great improvement over the prior art.

### DESCRIPTION OF THE DRAWINGS

Throughout the figures like reference numerals refer to identical structure, wherein:

FIG. 1 is a cross section view of a portion of the needle assembly;

FIG. 2 is a perspective view showing the entire needle assembly system in retracted position;

FIG. 3 is a cross section view of a portion of the needle assembly;

FIG. 4 is a perspective view showing the entire needle assembly system in engaged or deployed position;

FIG. 5 shows the context of the invention;

FIG. 6 shows a portion of the assembly that illustrates the detail of the retracted tip of the hemodialysis needle, as it would be during insertion into an access and during pull-out from an access;

FIG. 7 shows a portion of the assembly that illustrates the detail of the tip of the hemodialysis needle when in engaged position such as after successful insertion into a hemodialysis access, and during hemodialysis procedure

FIG. 8 shows a portion of the assembly that illustrates an alternative embodiment of the hemodialysis needle system for use with scar tissue track, in the retracted insertion position;

FIG. 9 shows a portion of the needle assembly that illustrates an alternative embodiment of the hemodialysis needle system for use with scar tissue track, in the deployed position; and,

FIG. 10 shows a portion of an alternative configuration of the needle tip.

### DETAILED DESCRIPTION

The needle system 30 consists of two concentrically positioned cylinders 1 and 2 that are each molded into their respective plastic bases.

The, outer cylinder 1 and inner cylinder 2 may both be fabricated as hollow hypotubes. Inner cylinder is positioned within an outer cylinder. Inner cylinder's outer diameter is such that it allows sliding of inner cylinder within the outer cylinder but provides a firm and snug fit.

The tip of the outer cylinder is beveled and sharp distal tip 20, allowing penetration through tissues. The tip of the inner cylinder

Has a mechanical feature 5 that exhibits “spring-like”, “shape-memory” properties. It has side walls forming limbs 24 that self-expand outwards when pushed and positioned outside of the outer cylinder 1. It deploys into a predefined “tulip-like” shape 5. When the end-portion of inner cylinder is expanded into a tulip like shape, it provides openings 6 on the sides of the inner cylinder 2, in addition to the circular opening 7 at the end of a cylinder as seen in FIG. 7 among others.

Materials that return to a predefined shape when external force is not applied are suitable to provide these properties. Such “shape-memory” materials show a defined degree of resilience and flexibility. Numerous bio-compatible polymers and alloys with such properties are available. Nitinol is a well known material suitable for the inner cylinder 2. Polymer materials may be used as well.

When system is in the retracted position, inner cylinder is retracted inside the outer cylinder. When retracted, the end-portion of the inner cylinder is pulled into the outer cylinder and its walls are compressed from a tulip-like shape into a cylindrical shape. Retracted position is required during the insertion of the needle system into vascular access, as well as, during the withdrawal of the needle from the vascular access.

Outer and inner cylinders are each molded into plastic base 3, and plastic base 4 respectively. Plastic bases are able to slide into each other. Proximal (male) base 4 can slide into a distal (female) base 3. Sliding the male base into the female base results in moving the end-portion of the inner cylinder from retracted to engaged position.

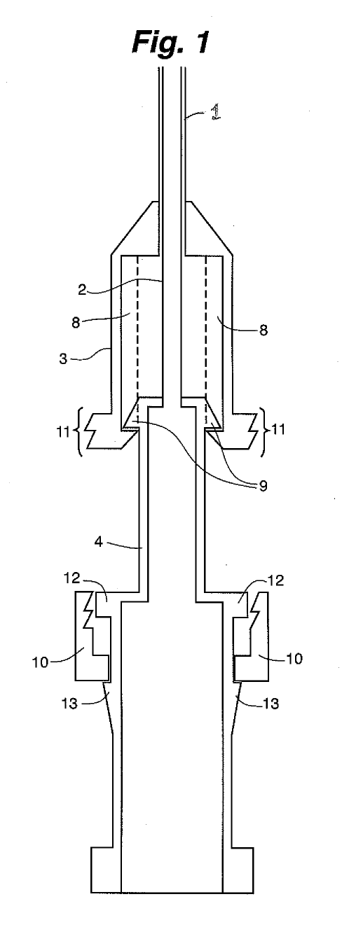

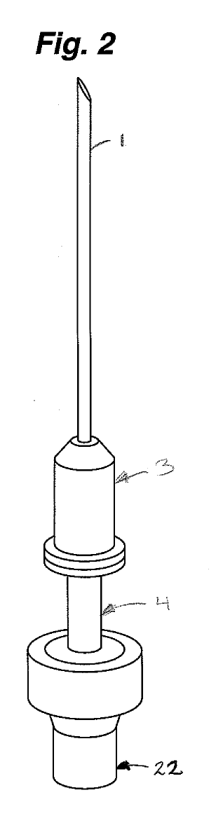

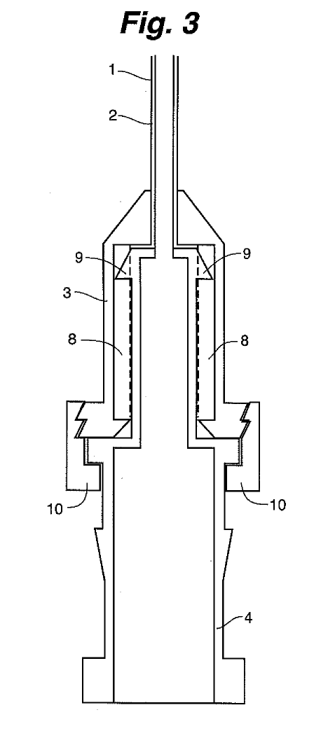

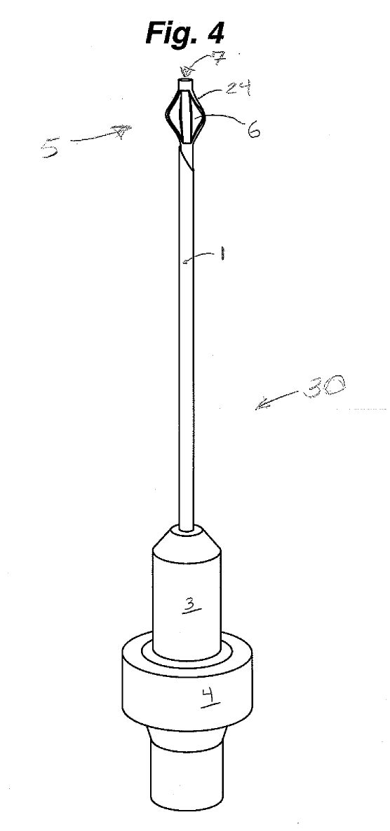

There are two grooves 8 in the luminal wall of the distal (female) base that run longitudinally with the long axis of the base. These accommodate corresponding two flanges elements seen twice in FIG. 1 and labeled according in the figure by reference numeral 9. These are formed on the proximal (male) base so that the bases do not rotate along their longitudinal axes relative to each other. This assures that the tip of the inner cylinder slides out and engages without rotating along its longitudinal axis relative to the outer cylinder. The other function of the two flanges 9 on the proximal (male) base is to serve as stops to prevent withdrawal of the proximal (male) base from the distal (female) base.

The proximal (male) base has a movable crown 10 that can be screwed into a thread 11 on the distal (female) base. This locks proximal (male) and distal (female) base together and secures the system in the engaged position. Prior to retraction of the system, crown has to be unscrewed so the proximal (male) base can be retracted from a distal (female) base. Proximal (male) base is shaped in such way that it has a rim 12 and two prominences 13 that serve as stops for the crown defining the range of move of the crown.

Plastic tubing 16 attaches to the proximal (male) base connector flange 22 and connects the needle system with the dialysis machine 21, while another piece of plastic tubing 19 connects to the needle assembly 30 on the other side of the AV access and the dialysis machine 21. Flexible needle wings (known to those skilled in the art) can be attached to a distal (female) base for better manipulation.

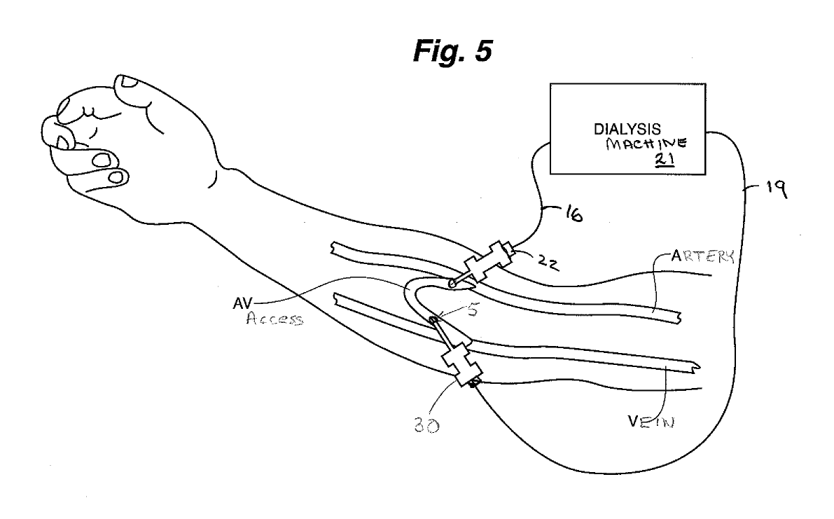

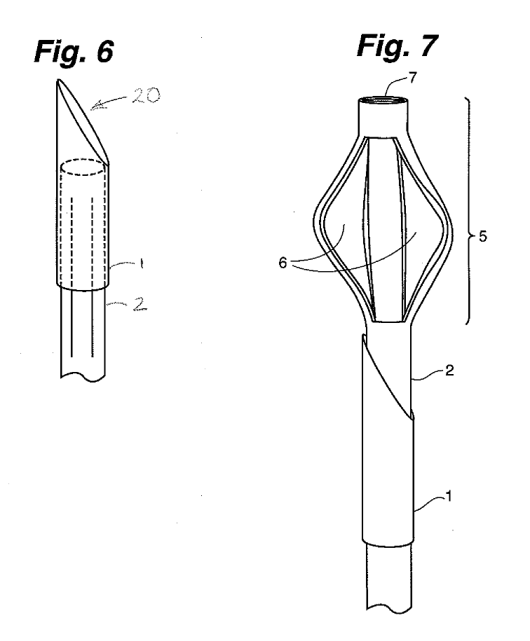

The tulip like blunt feature 5 seen deployed in some views (FIG. 5) reduces mechanical trauma inflicted by the needle to the patient's vascular access during the hemodialysis while the needle is positioned inside such access. When the end-portion of the inner cylinder is in the engaged position, it extends beyond the tip of the outer cylinder, and minimizes the contact of the sharp beveled tip of the outer cylinder with the wall of a dialysis access point.

The invention provides the system which decreases the chance of inadvertent needle withdrawal from vascular access during the hemodialysis. When the tip of the needle is engaged inside the vascular access, pulling the needle out of the access would require overcoming additional resistance imposed by the engaged end-portion of the inner cylinder.

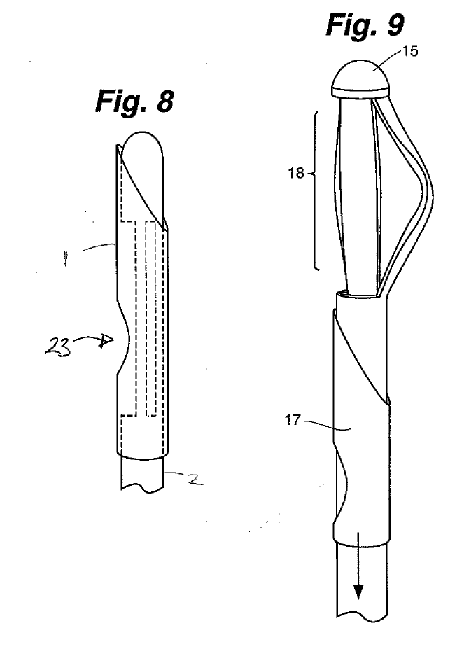

The invention further provides improved hemodynamic parameters of dialysis needle by improving the stability of blood flow during a hemodialysis. This is accomplished by the redundancy in openings available for a blood flow when the end-portion of the inner cylinder is in the engaged position. For example in the embodiment in FIG. 8 a side hole 23 allows the placement of the blunt tip 15 in an AV access with blood flowing into the side hole indicating proper placement in the AV access. In this embodiment the three legs form an enlarged opening 18 to support additional flow area. The side hole maybe covered by the outer cylinder 17 in the engaged position as seen in the figure (FIG. 9) or it may remain open providing additional flow area (not shown).

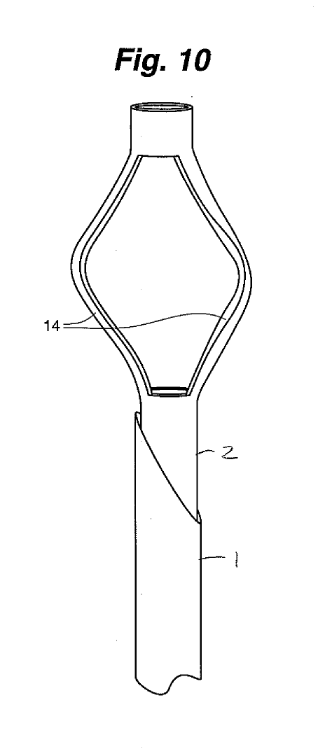

Although three and four limbed structures are shown in most of the figures forming the tulip shape, two limb embodiments are useful as well. In FIG. 10 Two identically shaped limbs (labeled twice in the figure as item 14) are seen with a large gap between them forming a flow passage.

What is claimed is:

1. A needle assembly for use in treating a dialysis patient comprising:
a first tubular needle having a tissue piercing tip and an interior lumen;
a second tubular shaft slideably mounted within said first tubular needle and movable from a first retracted position to a second activated position;
said second tubular shaft having distal slits that expand to form a fluid passageway in said second activated position, and which form a blunt tip distal of said piercing tip, and having a diameter larger than said first tubular needle.

2. The device of claim 1 wherein said second tubular shaft is formed from a shape memory material.

3. The device of claim 2 wherein said shape memory material is nitinol.

4. A needle assembly for use in perfusing a dialysis patient through a fistula comprising:
a first tubular needle having a tissue piercing tip and an interior lumen;
a second tulip shaped basket movable from a first retracted position within said first tubular needle, to a second activated position extending beyond said first tubular needle;
said second tulip shaped basket expanding to form an expanded atraumatic tip to prevent withdrawal of said assembly from a dialysis fistula.
